(19) United States
(12) Patent Application Publication
(10) Pub. No.: US 2011/0077545 A1
(43) Pub. Date: Mar. 31, 2011

(54) PORTABLE PNEUMOTACHOGRAPH FOR MEASURING COMPONENTS OF AN EXPIRATION VOLUME

(75) Inventor: Ruediger Eichler, Zellingen (DE)

(73) Assignee: FILT LUNGEN- UND THORAXDIAGNOSTIK GMBH, Berlin (DE)

(21) Appl. No.: 12/996,071

(22) PCT Filed: Jun. 3, 2009
(86) PCT No.: PCT/DE09/00791

§ 371 Date: Dec. 3, 2010

(30) Foreign Application Priority Data
Jun. 5, 2008 (DE) 10 2008 027 630.8
Jun. 5, 2008 (DE) 20 2008 007 748.6

(51) Int. Cl. A61B 5/08

(52) U.S. Cl. 600/538

(57) ABSTRACT

A portable pneumotachograph for determining components in an exhalation volume. The problem of extreme sensitivity of sensors to the temperature and moisture content of the gases, and cross-sensitivities to other potential components of the exhalation gas, is solved by providing a climate control chamber wherein a defined gas atmosphere can be set such that by means of the climate control chamber the relative humidity and/or the temperature and/or the composition of a sample and/or calibration gas can be set to predefined parameters.

The invention relates to a portable pneumotachograph for measuring components, in particular NO, in an expiration volume.

Nitrogen oxides and other gaseous compounds in the exhalation air are used for helping to assess the physical condition of human beings, because they are indicators for metabolic processes within the organism, disorders and diseases in human beings. Stationary instruments for a diagnostic gas analysis of the exhalation air are known and have been available on the market for a long time.

A portable gas analyser comprising an NO sensor is described in EP 1 439 781, wherein the patient exhales at a predetermined flow rate and pressure. One disadvantage of the instrument described therein is that no spirometric measurement data can be captured which enable a correlation between the captured measurement value and the corresponding affected area of the lung. Conditioning of the measurement or calibration gas, respectively, is done by means of a Nafion® hose, by means of which the humidity of the measurement or calibration gas is set to values of the ambient air.

EP 0 973 444 B1 discloses a device and a method for determining the NO content in the exhalation air using an initial device to determine the portion of nitrogen oxide over time during the exhalation phase, wherein during the commencing phase exhalation is done against no or only a small back-pressure and then against a resistance or back-pressure.

Also, instruments for pulmonary function analysis, i.e. spirometry, have been in practical use for years.

Spirometry is a method for testing the pulmonary function. Lung and respiratory volumes are measured and represented graphically in a spirogram. For capturing the lung volumes, a spirometer or a pneumotachograph is required.

The patient breathes into a breathing tube via a mouth piece, the nose being closed with a nose clip. Via a flow sensor, the spirometer electronically measures the air flow rate at which the patient inhales or exhales, and from this, the amount of the air respired per unit of time is calculated. The amounts of air moved during these breaths are reproduced graphically by the instrument. This also enables a direct comparison between the measurement values from different tests.

By measuring the air flow rate or exhalation rates and the lung volumes, a physician is able to diagnose diseases of the lung and to control their course. The following values can be measured using spirometry:

Tidal volume (TV): this corresponds to the volume of air inhaled or exhaled during normal breathing.
Inspiratory reserve volume (IRV): this is the volume which can be inhaled additionally after normal inhalation.
Expiratory reserve volume (ERV): this is the volume which can be exhaled additionally after normal exhalation.
Inspiratory capacity (IC): it is defined as the maximum volume which can be inhaled after normal exhalation.
Vital capacity (VC) is the maximum volume which can be exhaled after maximum inhalation.
Forced expiratory volume in one second (FEV1, Tiffeneau test) is the maximum volume which can be exhaled in one second after maximum inhalation.

These measurands help, for example, to distinguish between the two main groups of lung diseases:

Obstructive lung diseases: these are caused by a narrowing of the airways, e.g. due to asthma or COPD.
Restrictive lung diseases: in this case, the lung and/or thorax can be expanded only to a limited extent. Examples are pulmonary induration (pulmonary fibrosis), fluid accumulation in the pleura (pleural effusion) or a high diaphragm (diaphragm paresis).

During spirometry, the patient inhales and exhales via a mouth piece. The mouth piece is connected to a spirometer and in most cases provided with a bacteria filter. To capture the different measurands, the patient has to follow precisely the instructions of the examiner regarding inhalation and exhalation. Otherwise, incorrect values will be measured which could, in turn, lead to wrong conclusions during treatment. The examination thus depends on a good cooperation from the patient.

A further instrument for measuring NO in an exhalation volume is described in U.S. specification 6 010 459. Here, spirometry is used after capturing the values to be measured. The patient inhales synthetic air to which NO has been added and which has been subsequently humidified. When exhaling, the patient has to generate a defined pressure in the measuring instrument, ostensibly so that the velum closes on exhalation and no air from the nasopharynx, which could distort the measurement, enters the exhalation flow, where the NO concentration can be up to 100 times as compared to the pulmonary exhalation air. One disadvantage of this instrument is the fact that it is stationary, because for performing the measurements, the synthetic gas has to be used for inhalation.

A portable pneumotachograph for measuring components of an expiration volume is known from the German specification DE 20 2007 003 818.6. Here, a measurement of gaseous components takes place without prior conditioning of the measurement or calibration gas.

Moreover, the ATS/ERS guidelines (Exhaled breath condensate: methodological recommendations and unresolved questions. I. Horvath, J. Hunt and P. J. Barnes, On behalf of the ATS/ERS Task Force an Exhaled Breath Condensate, Eur Respir J 2005; 26: 523-548), published in 2005, for the first time provide an overall view of the methods for exhaled breath condensate diagnostics. Guidelines for NO measurements are set forth in the documents “ATS Workshop Proceedings: Exhaled Nitric Oxide and Nitric Oxide Oxidative Metabolism in Exhaled Breath Condensate: Executive Summary. Am J Respir Crit. Care Med. 2006 Apr. 1; 173(7):811-813” and “American Thoracic Society Documents: ATS Workshop Proceedings: Exhaled Nitric Oxide and Nitric Oxide Oxidative Metabolism in Exhaled Breath Condensate, Proc Am Thorac Soc Vol 3. pp 131-145, 2006”.

It is to be noted that the standardisation of sampling, sample storage and analysis needs yet to be improved. Sampling standardisation needs also to be done in future. Especially for analysis, the applied methods and their validation need to be given more attention.

Gas conditioning devices are also known from the art. In their simplest form, gases are humidified by injecting a fine mist of water, for example. Alternatively, gases are humidified by passing them through or along water-impregnated materials such as moist cloths, moist filters, etc.

Dehumidification of gases is achieved by silica gels or other dehydrating compounds. Moreover, gases can be dehumidified or humidified by means of drying and humidifying hoses. Such a hose is provided by the company Permapure and commercially available under the trade mark Nafion®.

For continuous drying of gases, the Nafion® is extruded as a tube. A Nafion® tube or a bundle of Nafion® tubes is enclosed by a housing, and a dry gas is made to stream against it in a counterflow. This maintains a water vapour partial pressure gradient between the gas and dry gas, and moisture is continually removed from the gas.

Humidification of gases is done using an analogous system. In this case, water is made to stream against the Nafion® in a counterflow. The gas can be tempered by means of a, thermostat in the water cycle. One disadvantage of this device is that an oversaturation and thus a condensation of water can occur in the gas-carrying Nafion® hose. The water entrained in the gas then leads to interferences in the measuring sensor.

With the mentioned systems, thus, only values of 0% or 100% RH are obtained reliably. One disadvantage of the humidifying and dehumidifying systems known from the art is that a conditioning of gases to values of, for example, 50%, 60% or 70% RH, if applicable, can be achieved with these systems only approximately.

The object of the invention is, therefore, to provide a pneumotachograph for measuring NO in an exhalation volume which is portable and easy to handle and permits a correlation between the captured measured data of one or more components of an exhalation flow and a pulmonary function test, wherein the analysis can be performed taking into account standardised guidelines and enables locating the focus of the disease.

When using electrochemical sensors, the problem is, in particular, the extreme sensitivity of these sensors to the temperature and moisture content of the gases. In addition, these sensors have cross-sensitivities to other potential components of the exhalation gas. With electrochemical sensors for determining NO, often a cross-sensitivity to NO₂ can be found. If the influence of the mentioned parameters is not taken into account when capturing the measurement values, non-comparable measurement values are obtained which will inevitably lead to wrong diagnoses.

In particular, precise gas conditioning is required for this sensor technology. Measuring sensors for determining gas components are, for example, used for flue gas analysis or in medical technology for respiration gas analysis. In particular, electrochemical sensors are used in gas analysis, which, however, are highly sensitive to the humidity and temperature of the gas.

Sensor types in which conditioning the measurement or calibration gas in the proposed manner will lead to more accurate measurement data are primarily electrochemical gas sensors, in particular NO, CO, CO₂ or O₂ sensors.

The object of the invention is achieved by providing a portable pneumotachograph for determining components in an expiration volume, comprising a processor, a PEEP valve attached on the pneumotachograph at the exhalation side, a filter attached on the pneumotachograph at the inhalation side for removing the portion of components to be determined in the inspiration air. At least one sampling unit is attached in or on the pneumotachograph tube and connected to a valve. At least one sensor is provided outside of the pneumotachograph tube for determining one of the components in the expiration volume. A pump is disposed downstream of the sensor for transporting the sample or calibration gas. A chemically and/or physically modified hose, which is enclosed by a climate control chamber for conditioning the sample gas and/or calibration gas flowing through the hose, is provided between the valve and a sensor. The sample and/or calibration gases to be conditioned can be conducted in the hose through the climate control chamber. In the climate control chamber a defined gas atmosphere can be set such that by means of the climate control chamber the relative humidity and/or the temperature and/or the composition of the sample and/or calibration gas flowing through the hose can be set to predefined parameters.

The sensor can be selected from the group of “electrochemical sensor, chemiluminescence sensor, enzymatic sensor, immunoassay sensor, NO sensor, O₂ sensor, H₂O₂ sensor, CO₂ sensor, CO sensor, sensor for biomarkers, ion mobility spectrometry (IMS) sensor” and/or a combined sensor system of said sensors. The corresponding sensor can be used depending on the component of the exhalation air to be analysed (NO, O₂). Also, multiple sensors can be provided for simultaneous measurement of different components.

The term “defined gas atmosphere” describes, in summary, the chemical and physical conditions in the climate control chamber. The climate control chamber is filled with gas. The gas has a defined humidity, temperature and composition. Preferably, it is a gas which is free from the components to be determined in the expiration volume. The temperature and the humidity set in the climate control chamber are preferably values which correspond to the ones of the optimum working range of the sensor and in which the measurement values are captured with only small degrees of variation, thus ensuring a stable and reproducible capturing of the measurement values.

The term “conduit carrying the sample and/or calibration gas” refers to the gas conduits leading from the sampling unit or the inlet of the calibration gas, respectively, to the conditioning hose and the gas conduits leading from the outlet of the climate control chamber up to the measuring chamber. The region of the measuring chamber and the gas conduit leading to the outlet are also covered by this term.

The humidity content and the temperature of the exhalate on leaving the lung are subject to variations and are to be regarded as not constant. Another change of these values which cannot be controlled occurs on entering the pneumotachograph tube. The causes for this are different in their nature. On the one hand, the exhalate can be cooled down, for example, if the inside of the pneumotachograph tube is cooler than the lung, for example due to an ambient temperature which is significantly lower as compared to the lung and thus a correspondingly lower temperature of the inspiration flow. In this case, this would effect a rise in the relative humidity in the pneumotachograph, as the relative humidity is a temperature-dependent value. A change of the mentioned values can also be due to a mixing with residual gases of different origins (residual exhalate of the previous breath, calibration gas residues, residues of the content, etc.). In this case, a reduction in the relative humidity could occur because of a “dilution effect” due to mixing with drier gases. Temperature variations can also occur.

From these exemplary cases it becomes clear that the gas samples to be measured have non-predictable parameters with regard to temperature and humidity, which, due to the sensitivity of the sensor to these parameters, will then lead to incomparable measuring value.

With the climate control chamber it is achieved that the humidity and the temperature of the gas sample taken from the expiration flow are set to the values in the climate control chamber. The gas sample reaching the sensor then has nearly constant values in terms of temperature and humidity. Consequently, the NO concentrations of different gas samples which are determined, for example, within this temperature and humidity window are comparable, since the parameters influencing the measurement are maintained constant.

The principle of conditioning is based on evening out concentration and temperature gradients. While flowing through the hose, the differences in said parameters between the climate control chamber and the hose volume will even out, whereby the moisture content and the temperature will equalise.

By means of the climate control chamber the relative humidity of the gas flowing through the hose can be set to values of 0% to 100% RH, according to the value in the climate control chamber. At the same time, the gas is tempered to the temperature set in the climate control chamber.

The hose for conditioning the sample gas and/or calibration gas flowing through it, in the following referred to as conditioning hose, has chemical and/physical properties which allow an equalisation of humidity and temperature. Preferably, the hose, which is chemically and/or physically modified in its properties, is permeable to water vapour. The membrane properties of the hose material, therefore, have permeable or semi-permeable properties in terms of the exchange of humidity and, if applicable, also with regard to interfering compounds, while at the same time having low heat insulating properties. Reference is made in particular to a Nafion® hose available from PermaPure.

The volume of the climate control chamber is preferably several times larger than the hose volume so as to minimise as far as possible the influence of the humidity and temperature of the sample gas or calibration gas on the values in the climate control chamber and to effect an equalisation of the parameters of the sample or calibration gas to the values in the climate control chamber. The ratio between the climate control chamber volume and the hose volume is preferably 200/1 to 4/1, preferably 5/1.

The climate control chamber is preferably a space closed off from the environment through which the conditioning hose is passed. For this purpose, at least two connections or two lead-throughs are provided in the wall of the climate control chamber as an inlet and outlet. The connections or lead-throughs, respectively, are sealed from the interior of the climate control chamber so as to prevent an exchange of the gases between the interior and the hose volume or the environment, respectively.

In the climate control chamber, a humidity sensor or a temperature sensor, or both, can be provided. Via these sensors, the condition in the climate control chamber is checked, and if there are any deviations from the set values, the deviation is signalled. The analysis of the set/actual values occurs preferably in a process-controlled manner. The signal can be an optical or an acoustic signal. Moreover, the measurement procedure can be interrupted on the occurrence of a deviation. Also, there can be a process-controlled correction of the state parameters in the climate control chamber if the actual values deviate from the set values. For this purpose, additional means are provided with which a corrective adjustment of the actual values can be performed.

In addition, the conduit carrying the sample and/or calibration gas can be equipped with one or more temperature and/or humidity sensors before or after the climate control chamber by means of which the actual state of the sample and/or calibration gas can be determined with regard to its temperature and/or moisture content before or after the climate control chamber.

Sensors which are provided after the climate control chamber in the conduit carrying the sample serve, among other things, for checking the actual parameters of the sample or calibration gas after climatisation and, if applicable, for detecting a deviation from the set value defined by the climate control chamber. If a deviation is detected, the gas atmosphere in the climate control chamber can be corrected using the actual values and a gas atmosphere can be set by means of which the desired set values are obtained. A correction of the temperature and/or humidity of the sample and/or calibration gas can also be performed, however, by adjusting the flow which determines the retention and contact time of the sample or calibration gas, respectively, with the gas atmosphere in the climate control chamber. Preferably, the correction of the gas atmosphere and/or the flow is also performed in a process-controlled manner via appropriate control signals generated by the temperature or humidity sensor(s).

Also, the actual data of the temperature and the moisture content of the sample or calibration gas, respectively, can be used to correct the measurement values based on a mathematical correlation between the actual and the set values.

The climate control chamber can be equipped with a means for setting a defined relative humidity in the climate control chamber. In the simplest case, a water-storing medium is placed into the climate control chamber. This can be a water-impregnated cotton pad or a water-storing gel by means of which a saturation of the gas in the climate control chamber to 100% RH is achieved due to evaporation of the water. The reverse case of 0% RH can also be achieved by placing a water-binding medium, preferably silica gel, into the climate control chamber.

In a particular embodiment the means for setting the humidity is a device having two containers, each containing a gas of a defined relative humidity, wherein the moisture contents of the two gases are different from one another. Preferably, one container contains a gas of 0% RH and the other container a gas of 100% RH. The containers can be realised such that they can be tempered. The containers are connected to the climate control chamber via a common controllable mixing valve, and via the mixing valve the relative humidity of the gas in the climate control chamber can be set in a process-controlled manner by mixing the two gases from the containers based on measurement values of the humidity sensors which are converted into control signals. Preferably, after the mixing valve a mixing chamber is provided additionally having a corresponding sensor system for determining the temperature and humidity of the mixed gas. The mixed gas is passed on to the climate control chamber only when the set values have been reached in the mixing chamber.

In a preferred embodiment, the climate control chamber is equipped with a means for tempering the climate control chamber. Preferably, it is controllable by means of the processor. In particular, such a means is a Peltier element which can be used for both heating and cooling.

For example, a storage reservoir containing conditioned chamber gas can also be provided, the storage reservoir being connected to the climate control chamber. On the indication or detection of a deviation of the actual values from the set values, the gas atmosphere is then exchanged.

The volume flow of the gas inside the hose is 1 to 20 ml/s, preferably 2 to 3 ml/s.

The dimensions of the hose, such as length, internal diameter, wall thickness, and the volume flow are selected such that the gas flowing through the hose, on entering the measuring chamber of the sensor, has a humidity and temperature corresponding to the gas atmosphere in the climate control chamber.

The valve attached in or on the pneumotachograph tube is preferably a static three-way valve. By means of the three-way valve, switching back and forth between the calibration and sample gas can be done without the necessity of any structural alterations to the pneumotachograph. The calibration gas is also set to sensor-optimised values in the climate control chamber.

In a preferred embodiment, in the conduit carrying the calibration gas an additional filter is provided before the valve for removing components from the calibration gas which interfere with the calibration, the filter being dimensioned such that its size and filter effectiveness are appropriate for the amount of calibration gas and the flow conditions at the sensor. Preferably, it is an activated carbon filter. In one embodiment, the activated carbon filter can contain potassium permanganate.

The electrochemical nitric oxide sensor has a measuring rage of 0 to 5000 ppb, preferably 0 to 3000 ppb, more preferably 0 to 1000 ppb, in particular 0 to 500 ppb.

The conditioning hose can be filled partially or entirely with a filter material and/or the internal wall of the hose can be covered with a chemically or physically active film, which removes interfering compounds negatively affecting the measurement, in particular NO₂, from the calibration or sample gas. The removal can be achieved by chemical conversion, adsorption or dissolving the compound in the film.

The core of the portable pneumotachograph for determining components of an expiration volume is a pneumotachograph to which preferably a replaceable mouth piece and/or a bacteria filter are attached. The patient inhales and exhales through the pneumotachograph. At the inhalation side a filter is attached on the pneumotachograph for filtering out of the ambient air the component to be measured. A sampling unit is provided in the pneumotachograph in the tube, for example in the centre of flow, or in the tube wall in the exhalation direction of flow after the lamellae or grille being present in the pneumotachograph tube for generating a flow resistance.

The sampling unit is connected to the valve via a feeding conduit. The sensor is provided outside the pneumotachograph tube and connected to the valve via another conduit which can consist partially or entirely of the conditioning hose. Multiple sampling units can also be provided which can be controlled independently from one another.

In a particular embodiment the pneumotachograph is equipped with multiple measuring units, wherein one measuring unit consists of a sampling unit, a valve, a feeding conduit leading to the sensor and the sensor itself. Thus, apart from sensors with gas conditioning, sensors without gas conditioning can also be provided in the pneumotachograph. Also, depending on the sensor type, a decision can be made by a process-controlled circuit between feeding the gases with or without conditioning of the gas, wherein switching back and forth between feeding them to the sensor without climatisation and feeding them to the sensor with climatisation can be done without having to provide a separate climate control chamber for each sensor.

Sensor types in which conditioning the sample or calibration gas in the proposed manner leads to more accurate measurement data are, in particular, NO, CO, CO₂ or O₂ sensors. This list is not exhaustive.

According to the inspiration or expiration or a partial inspiration or partial expiration volume or flow passing through the pneumotachograph tube, the valve can have the states of “open” or “closed”, wherein the valve can be driven by the processor. Hereby it is achieved that a sampling or measurement is performed only when defined volume flows or partial volume flows of the inspiration or expiration pass through the pneumotachograph tube. The point of time at which a measurement has to take place is calculated via the flow/volume correlation determined by the pneumotachograph, which is connected to a processor to which also the valve is connected and via which the valve can be driven. Since sampling is done in a process-controlled manner directly from the pneumotachograph, any delay or discordance between the flow measurement and sampling is primarily excluded or compensatory corrections are performed, respectively.

Exhalation can be done against an expiratory resistance, which is generated by the PEEP valve attached on the pneumotachograph tube at the exhalation side. The expiratory resistance is preferably 5 to 20 cm H₂O and effects an increase in the medium pressure of the airways and the functional residual capacity.

By means of the PEEP valve it is achieved that the de-airing of the lung or the pulmonary alveoli, respectively, is more uniform also in the case of a narrowing of the airways, secretion in the airways or other ventilation disorders (distribution disorder). This measure is a prerequisite for a reproducible repetition of the breathing manoeuvres and a largely undisturbed emission of the components of the exhalation air during measurement.

In a particular embodiment the PEEP valve is provided such that it can be removed. Thus, the portable pneumotachograph can also be used for measuring spirometric data without having to provide another spirometer. While recording the spirometric measurement data, the measuring sensor is preferably put out of operation or switched off.

In a particular embodiment the PEEP valve is a double valve which limits the flow between a minimum flow and a maximum flow. In other words, the valve opens on reaching a first exhalation pressure being in correlation with the minimum flow, whereby the flow rate inside the pneumotachograph jumps up from the value of “zero” to the flow rate of the minimum flow. The patient can now exhale at a defined flow. On exceeding a second exhalation pressure, which is larger than the first exhalation pressure and which correlates with the maximum flow, the valve closes again and the value of the flow rate jumps down to the value of “zero”. The patient is now prevented from exhaling. Exhalation is possible only between the minimum flow and the maximum flow. Such a PEEP double valve has, for example, at the entry side (at the pneumotachograph side) a spring-loaded check valve and at the exit side (environment) a pressure valve, wherein the spring-loaded check valve opens only on reaching or exceeding, respectively, a first pressure generated at the entry side and the pressure valve closes at the exit side on exceeding a higher stagnation pressure generated at the entry side which is larger than the first pressure (opening pressure). In other words, the PEEP valve opens at its entry opening only on overcoming a first defined pressure resistance. From this time on, its exit opening is open. If the flow is increased, the pressure acting on the check valve also increases and the check valve spring is compressed further and at the same time pushes towards the exit opening. At the backside between the closure of the check valve and the spring a taper is attached, for example, which, on compression of the spring, engages the exit opening and successively closes it. With this type of PEEP valve, the expiratory flow and the expiratory resistance can be limited between a minimum value and a maximum value, each of which can be set.

For checking the flow, the portable pneumotachograph has an optical or acoustic check, by means of which the patient can check and adjust his or her exhalation. The optical check of the expiration flow can be selected from the group of “y-t graph, bar chart, light-emitting diode display having one or more light-emitting diodes”. The acoustic check can be a beep sound or a sound changing in loudness or frequency.

In a preferred embodiment, a valve or flap is provided between the filter attached on the pneumotachograph at the inspiration side, the filter being provided such as to be replaceable, and the pneumotachograph tube, the valve or flap closing the entry opening of the pneumotachograph during expiration. Hereby it is achieved that the patient breathes only against the expiratory resistance effected by the PEEP valve. The valve or flap preferably closes self-actingly. Such a valve can be a check valve, a shut-off valve or a simple flap which opens into the pneumotachograph tube. No restrictions are provided in terms of the component parts, however.

The portable pneumotachograph can have one or more collection containers impervious to gases for collecting samples and/or respiration volumes of several breaths. By means of these, several breaths can be combined or samples can be kept until they are analysed.

The surfaces of the conduits carrying the sample or the air ducts of the pneumotachograph according to the invention can be modified and can be such that membranes, liquid films are applied on them or inserts of porous layers or membranes are worked into them such that certain components of the sample gases are retained or chemically bound in the layers, respectively. This list is exemplary and not restrictive. In this way, substances or chemicals, respectively, interfering with the analysis, for example compounds causing cross-sensitivities at the sensor, such as NO₂, can be physically or chemically bound from the expiration flow or inspiration flow so to be able to ensure an interference-free analysis. On the other hand, free radicals can be deactivated by chemical conversion in or at the modified surfaces and the sample thus be stabilised.

The portable pneumotachograph can be additionally equipped with a purging apparatus allowing the sensor and/or the sampling unit (sampling unit, valve, feeding conduits, hose) to be purged with a gas, selected from the group of “component-free air, synthetic air, gases prepared for the purpose of calibration” or a combination of the mentioned gases, and thus be cleared from the exhalation air to increase the accuracy of the measurements. For this purpose, a pump can be provided which is connected, for example, to the filter attached on the pneumotachograph at the inspiration side via a, preferably separate, conduit and which pumps ambient air through the filter into the sampling and sensor area or into one of the two areas. Moreover, one or more connections can be provided for connecting a pressurised or unpressurised purge gas bottle.

In another embodiment a calibration of the sensor can be done with a gas, selected from the group of “component-free air, synthetic air, gases prepared for the purpose of calibration” or a combination of the mentioned gases, after one or a number of measurement units. The calibration values of the mentioned gases can be set on the instrument individually.

The component-free air can be produced by pumping indoor air at the inspiration side through the filter attached on the pneumotachograph. Subsequently, this purified air is conditioned in the climate control chamber and subsequently conducted over the sensor.

To reduce the risk of a faulty measurement by the admixture of nasal air, the following measures can be provided:
wearing a nose clip during inspiration
exhaling at a constant flow or a flow greater than zero,
   because, if the flow stops during the exhalation manoeuvre, this brings air from the nose into the pharynx.

A constant flow is a flow having a maximum deviation of +/−10% from the average value. The constant flow of the expiratory air can be preferably 10 to 500 ml/s, in particular 45 to 55 ml/s. Preferably it is 50 ml/s. It can also be varied, however.

The expiratory flow has to be maintained constant for a duration of 1 to 30 s, preferably 2 to 10 s, in particular 4 to 6 s.

The flow of 50 ml/s should be maintained within a range of +/−10% for 4 s in the case of children younger than 12 years or 6 s in the case of children older than 12 years and adults. This corresponds to a total of about 300 ml of air at a flow of 50 ml/s.

Adjustment to the flow rates and pressures necessary for the analysis of one or more components of the exhalation air, for example because of required framework conditions due to legal provisions or guidelines, is done by selecting and using a PEEP valve determining the required flow and pressure or, in the case of a controllable PEEP valve, by setting it to the required parameters.

During this plateau, the NO measurement value is maintained in a range corresponding to the guidelines of the American Thoracic Society (ATS) and the European Respiratory Society (ERS), which have been adopted by the ATS in December 2004 and by the ERS in June 2004.

The measurement is to be repeated. A measurement is subject to the ATS/ERS guidelines if at least two breathing manoeuvres meet the criteria.

In another embodiment, a partial or the entire expiratory volume can be collected in one or more gas-impervious collection receptacles, preferably a gas bag. Alternatively, this task can also be performed by the volume of the pneumotachograph.

In a preferred embodiment, the pneumtachograph tube has a valve or flap in the inlet for the inspiratory air, which can be closed, and multiple outlets for the expiratory air, wherein in the outlets for the expiratory air outlet valves are provided. By means of the flow meter the volume flow of the expiratory air is determined and, based on this value, it is split up into initially theoretical values of partial volume flows by means of a processor-aided calculation. To each of these partial volume flows a zone of the respiratory tract can be assigned. The valve or flap in the inlet for the inspiratory air and/or the outlet valves can be controlled by the processor, wherein the operational states of “closed” (a) or “open” (b) of the valve or flap in the inlet and/or of the outlet valves can be set according to a particular partial volume flow of the volume flow of the expiratory air.

Thus, by means of the value of the expiration volume an allocation to partial volume flows of an expiration volume exhaled successively in time can be performed, wherein certain regions and zones of the respiratory tract can be assigned to these partial volume flows. By the operation, diseases and disorders of the respiratory tract can be located.

In another embodiment it is provided that a measurement of components of the expiration volume takes place only when a defined partial expiration volume passes through the pneumotachograph tube, in particular the sampling unit. By capturing the spirometric measurement data it can be calculated via the process at which point in time a partial volume flow of x, originating in the area Y of the lung, where, for example, the focus of a disease is assumed to be located, passes through the sampling unit in particular. A measurement of the components only of this partial volume flow can then be started.

Under the condition of standards, such as the ATS/ERS guidelines, only measurement values can be analysed and are representative which have been obtained at defined measurement conditions. For this purpose it is provided that a measurement of components of the expiration volume takes place only on the occurrence of the predefined parameter or parameters of the expiration volume of “overcoming the expiratory resistance” and/or “constant expiratory flow” and/or “duration of the expiratory flow”. If theses parameters are not reached or not maintained for a sufficient amount of time, no measurement value is recorded, i.e. the sensor signal is not analysed by the processor. If there is no signal acceptance or if a condition has not been met after initiating the sensory measurement, a measurement value which has been determined at non-standard conditions can be marked and outputted as such.

Preferably, the values of the parameters “overcoming the expiratory resistance” and/or “constant expiratory flow” and/or “duration of the expiratory flow” can be set individually and/or or can be retrieved from a storage medium, preferably the processor, according to the patient group and/or the condition of the expiratory tract of the patient.

In the following, the invention will be explained in more detail by way of two exemplary embodiments:

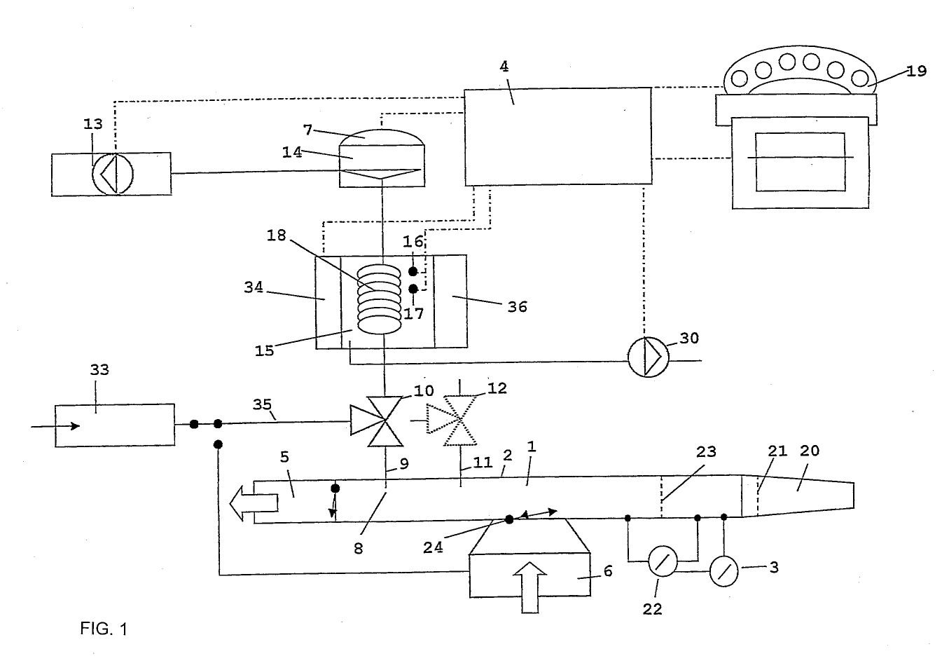

FIG. 1: shows a schematic representation of the portable pneumotachograph

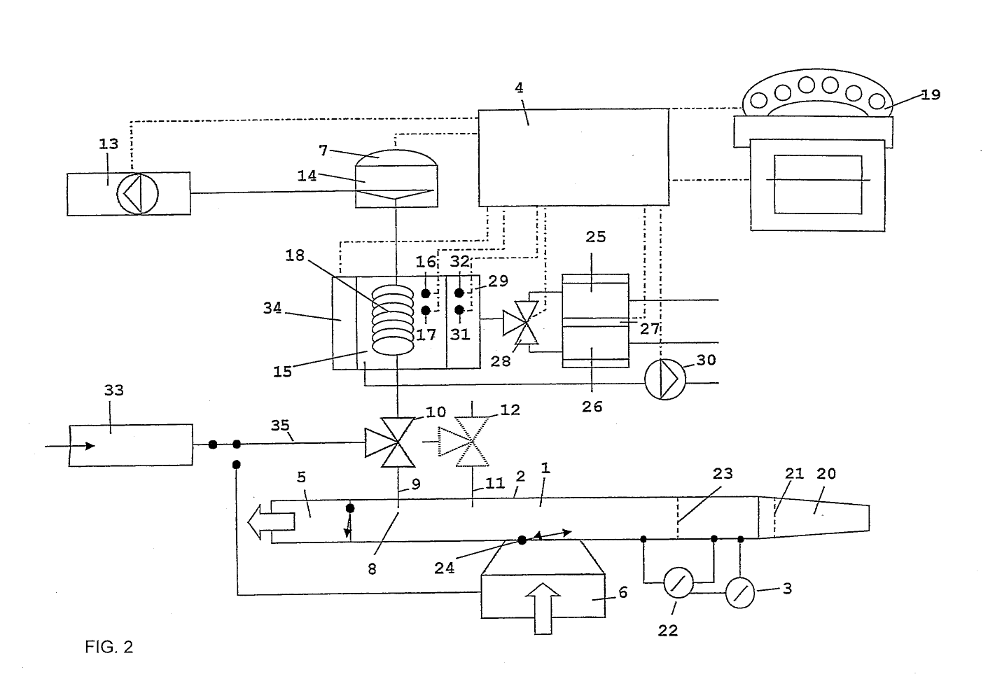

FIG. 2: shows a schematic representation of an alternative embodiment

In the figures, continuous lines represent conduits for carrying the sample and/or calibration gas, dotted-and-dashed lines represent electronic connections for data and signal transfer.

FIG. 1 shows a portable pneumotachograph for determining components of an expiration volume, comprising a pneumotachograph 1 having a pneumotachograph tube 2, a means for pressure measurement 3 and a processor 4, a PEEP valve 5 attached on the pneumotachograph 1 at the exhalation side, a filter 6 attached on the pneumotachograph 1 at the inhalation side for removing the portion of components to be determined in the inspiration air, and a sensor 7. A sampling unit 8 having a conduit 9, which passes through the wall of the pneumotachograph tube 2, is provided in the duct of the pneumotachograph tube 2. The sampling unit 8 is connected to the valve 10, in this case a three-way valve, via the conduit 9.

In addition, a second sampling unit 11, also with a three-way valve 12, is provided, which (not shown for the sake of clarity) is connected to a second sensor. The feeding conduit leading to the sensor can also be equipped with a climate control chamber. If a climate control chamber is not necessary due to the selected sensor type, it is dispensable and the gas is conducted to the measuring chamber without being conditioned.

Downstream a pump 13 is provided at the sensor 7 for transporting the calibration or sample gas. Between the valve 10 and the measuring chamber 14 of the sensor 7 there is a climate control chamber 15, through which the conditioning hose 18 is passed. A water-impregnated cotton pad 36 is placed into the climate control chamber 15, by means of which the gas atmosphere in the climate control chamber 15 is saturated with moisture. The climate control chamber 15 is equipped with a temperature sensor 16 and a humidity sensor 17, which are connected to the processor 4. The climate control chamber 15 has a Peltier element 34 for tempering the climate control chamber 15. As an alternative to the Peltier element, a resistance heating element in the form of a heating foil element can be used for tempering.

The pneumotachograph tube 2 has an optical check 19 of the expiration flow. The mouth piece 20 is equipped with a bacteria filter 21. As per standard, the pneumotachograph 1 has an electric manometer 22, which measures the pressure difference before and after the lamellae 23.

Between the filter 6 attached on the pneumotachograph 1 at the inhalation side and the pneumotachograph tube 2 an inspiratorily effective flap 24 is provided, which closes the entry opening of the pneumotachograph 1 on expiration.

Continuous lines represent conduits, dotted-and-dashed lines represent electronic connections for data and signal transfer.

FIG. 2 shows an alternative embodiment of the portable pneumotachograph 1. The climate control chamber 15 is equipped with a means for setting a defined relative humidity in the climate control chamber. This device consists of two containers 25, 26, each containing a gas of a defined relative humidity, wherein the moisture contents of the two gases are different from one another. The one container 25 contains a gas of 0% RH and the other container 26 a gas of 100% RH. The containers 25, 26 can be tempered by means of a heating element 27. The containers 25, 26 are connected to the climate control chamber 15 via a common mixing valve 28 which can be controlled via the processor 4. By means of the mixing valve 28 the relative humidity of the gas in the climate control chamber 15 can be set in a process-controlled manner by mixing the two gases from the containers 25, 26 based on measurement values of the humidity sensor 17 which are converted into control signals.

After the mixing valve 28 a mixing chamber 29 can be provided having a temperature 32 and humidity sensor 31 for determining the temperature and humidity of the mixed gas. Alternatively, the mixing chamber 29 and the climate control chamber 15 can form a unit.

The mixed gas is transported into the climate control chamber 15 by means of the mixing pump 30. The climate control chamber 15 is also equipped with a temperature 16 and a humidity sensor 17, which are connected to the processor 4. Similarly, the mixing valve 28 and the corresponding mixing pump 30 are connected to the processor 4. Moreover, the climate control chamber 15 has a Peltier element 34 for tempering the climate control chamber.

In this embodiment, in the conduit 35 carrying the calibration gas an additional filter 33 is provided before the valve 10 for removing components from the calibration gas which interfere with the calibration, the filter being dimensioned such that its size and filter effectiveness are appropriate for the amount of calibration gas and the flow conditions at the sensor.

### LIST OF REFERENCE NUMERALS

1 pneumotachograph
2 pneumotachograph tube
3 a means for measuring the pressure
4 a processor
5 PEEP valve
6 filter
7 sensor
8 sampling unit
9 conduit
10 valve
11 second sampling unit
12 second valve
13 pump
14 measuring chamber of the sensor
15 climate control chamber
16 temperature sensor climate control chamber
17 humidity sensor climate control chamber
18 conditioning hose
19 optical check
20 mouth piece
21 bacteria filter
22 manometer
23 lamellae, screen or sieve
24 inspiratorily effective flap
25 container
26 container
27 tempering element
28 mixing valve
29 mixing chamber
30 mixing pump
31 humidity sensor
32 temperature sensor
33 calibration gas filter
34 tempering element
35 calibration gas conduit
36 cotton pad

1. A portable pneumotachograph for determining components in an expiration volume, comprising a processor (4), a PEEP valve (5) attached on the pneumotachograph (1) at the exhalation side, a filter (6) attached on the pneumotachograph (1) at the inhalation side for removing the portion of components to be determined in the inspiration air, at least one sampling unit (8) attached in or on the pneumotachograph tube (2), a valve (10) connected to the sampling unit (8), at least one sensor (7) provided outside of the pneumotachograph tube for determining one of the components in the expiration volume, and a pump (13) disposed downstream of the sensor (7), wherein a chemically and/or physically modified hose (18), which is enclosed by a climate control chamber (15) for conditioning the sample gas and/or calibration gas flowing through the hose (18), is provided between the valve (10) and the sensor (7), and wherein the sample and/or calibration gases to be conditions can be conducted in the hose through the climate control chamber, and in the climate control chamber (15) a defined gas atmosphere can be set such that by means of the climate control chamber (15) the relative humidity and/or the temperature and/or the composition of the sample and/or calibration gas flowing through the hose (18) can be set to predefined parameters.

2. A portable pneumotachograph according to claim 1, wherein the relative humidity of the gas flowing through the hose (18) can be adjusted to values of 0% to 100% RH.

3. A portable pneumotachograph according to claim 1, wherein the chemically and/or physically modified hose (18) is permeable to water vapour and is preferably a Nafion® hose.

4. A portable pneumotachograph according to claim 1, wherein a humidity sensor (17) and/or temperature sensor (16) is provided in the climate control chamber (15).

5. A portable pneumotachograph according to claim 1, wherein the volume flow of the gas in the hose (18) is 1 to 20 ml/s, preferably 2 to 3 ml/s, and can be set as a function of the purge time of the measuring chamber (14) of the sensor (7) and the feeding system (8, 9, 10, 18, 33, 35) leading to the sensor (7).

6. A portable pneumotachograph according to claim 1, wherein the climate control chamber (15) is equipped with a, preferably controllable, means for controlling the temperature in the climate control chamber (34).

7. A portable pneumotachograph according to claim 6, wherein the means for controlling the temperature (34) is a Peltier element or a resistance heating element.

8. A portable pneumotachograph according to claim 1, wherein the climate control chamber (15) is equipped with a means for setting a defined relative humidity in the climate control chamber (15).

9. A portable pneumotachograph according to claim 8, wherein the means for setting the humidity is a water-storing medium, preferably a water-impregnated cotton pad, or a water-binding medium, preferably of silica gel.

10. A portable pneumotachograph according to claim 8, wherein the means for setting the humidity is a device having two containers (25, 26), each containing a gas of a defined relative humidity, wherein the moisture contents of the two gases are different from one another, wherein the container (25, 26) are connected to the climate control chamber (15) via a common controllable mixing valve (28), and by means of the mixing valve (28) the relative humidity of the gas in the climate control chamber (15) can be set in a process-controlled manner by mixing the two gases from the containers (25, 26) based on measurement values of the humidity sensor (17) which are converted into control signals.

11. A portable pneumotachograph according to claim 10, wherein a heating element (27) is provided for controlling the temperature in the containers (25, 26).

12. A portable pneumotachograph according to claim 10, wherein between climate control chamber (15) and mixing valve (28), a mixing chamber (29) is provided having a humidity sensor (31) and a temperature sensor (32).

13. A portable pneumotachograph according to claim 1, wherein the valve (10) is a static three-way valve.

14. A portable pneumotachograph according to claim 1, wherein the sensor (7) is selected from the group of “electrochemical sensor, chemiluminescence sensor, enzymatic sensor, immunoassay sensor, NO sensor, O₂ sensor, H₂O₂ sensor, CO₂ sensor, CO sensor, sensor for biomarkers, ion mobility spectrometry (IMS) sensor” and/or a combined sensor system of said sensors.

15. A portable pneumotachograph according to claim 14, wherein the sensor (7) is an electrochemical nitric oxide sensor, preferably having a measuring rage of 0 to 5000 ppb, preferably 0 to 3000 ppb, more preferably 0 to 1000 ppb, in particular 0 to 500 ppb.

16. A portable pneumotachograph according to claim 1, wherein the pneumotachograph tube (2) has a valve or flap (24) in the inlet for the inspiratory air which can be closed, wherein the pneumotachograph tube has multiple outlets for the expiratory air, wherein outlet valves are provided in the outlets for the expiratory air, wherein by means of the flow meter the volume flow of the expiratory air can be measured and, based on this value, it can be split up into theoretical values of partial volume flows by means of a processor-aided calculation, wherein the partial volume flows can be assigned to different zones of the respiratory tract, wherein the valve or flap (24) in the inlet for the inspiratory air and/or the outlet valves can be driven and controlled by the processor (4), and wherein the operational states of “closed” (a) or “open” (b) of the valve or flap (24) in the inlet for the inspiratory air and/or the outlet valves can be set according to a particular partial volume flow of the volume flow of the expiratory air.

17. A portable pneumotachograph according to claim 1, wherein the PEEP valve (5) is a double valve which limits the flow between a minimum flow and a maximum flow.

18. A portable pneumotachograph according to claim 1, wherein the chemically and/or physically modified hose (18) for removing compounds in the sampling or calibration gas interfering with the sensory measurement is filled entirely or partially with a filter material and/or the internal wall of the hose is covered with a chemically or physically active film.

19. A portable pneumotachograph according to claim 1, wherein a processor (14) is provided for controlling the climate control chamber (15) and/or the mixing chamber (29).

20. A portable pneumotachograph according to claim 1, wherein in the conduit carrying sample and/or calibration gas, one or more temperature and/or humidity sensors are provided before or after the climate control chamber.
